(19) United States
(12) Patent Application Publication
(10) Pub. No.: US 2015/0314136 A1
(43) Pub. Date: Nov. 5, 2015

(54) PHOTO-MEDICINE SYSTEM AND METHOD

(71) Applicant: Illumitex, Inc., Austin, TX (US)

(72) Inventors: Daniel Marvin Watkins, Houston, TX (US); Dung Tien Duong, Bee Cave, TX (US); Charles Alicea, Austin, TX (US); Gretchen Heber, Austin, TX (US); Janet Lee Hammelef, Austin, TX (US); Nicholas Flynn Jameson, Cedar Park, TX (US)

(21) Appl. No.: 14/702,415

(22) Filed: May 1, 2015

Related U.S. Application Data
(60) Provisional application No. 61/987,369, filed on May 1, 2014.

(51) Int. Cl. A61N 5/06

(52) U.S. Cl. CPC A61N 5/0616; A61N 2005/0626; A61N 2005/0652; A61N 2005/0663

(57) ABSTRACT

A photo-medicine device may include a housing having: a mounting member and an application member including an aperture. An LED array having at least one LED configured to emit light through the aperture at a first wavelength and at least one LED configured to emit light through the aperture at a second wavelength may be mounted to the mounting member. The LED array may be in thermal communication with the mounting member such that the housing functions as a heat sink for the LED array. In some embodiments, the first wavelength comprises approximately 415 nm and the second wavelength comprises approximately 660 nm. In some embodiments, the housing has a heat dissipation surface area of at least three square inches per LED watt.

### CROSS-REFERENCE TO RELATED APPLICATION(S)

This application is a conversion of, and claims a benefit of priority from U.S. Provisional Application No. 61/987,369, filed May 1, 2014, entitled “PHOTO-MEDICINE SYSTEM AND METHOD,” which is fully incorporated by reference herein.

### TECHNICAL FIELD

Embodiments described herein are related to photo-medicine systems and methods.

More particularly, embodiments relate to a photo-medicine device having a light-emitting diode (LED) array useful for treating acne and building collagen.

### BACKGROUND

Acne vulgaris is one of the most common skin conditions to affect humans, with 70% of adolescents developing acne and 40 to 50 million people affected in the U.S. Nearly 85% of all people have acne at some point in their lives.

Acne is a problem for numerous reasons: unsightliness can cause extremely low self-esteem and self-confidence; unsightliness can cause others to respond poorly to acne sufferers; acne can lead to harmful skin infections; and unattractive, permanent scarring can result from acne.

Other skin disorders, too, can cause significant, undesirable psychosocial affects. Wrinkles, blemishes, age spots and uneven pigmentation are considered by many cultures to be unattractive and worthy of eradication.

Specific wavelengths available in LEDs have been proven to kill the acne vulgaris bacteria. Other wavelengths have been identified as effective in building collagen and increasing cell turnover, eliminating fine wrinkles, blemishes, age spots, and uneven pigmentation in skin.

While devices are known for application of such wavelengths for therapeutic purposes, they are relatively bulky and are not provided in a common compact package. Indeed, difficulties arise when an LED array capable of delivering light at wavelengths of suitable intensity is shrunk to a desirably compact size. In particular, heat generated by such an array can cause damage to the device itself as well as the skin of the patient being treated.

Accordingly, it would be desirable to provide a photo-medicine device capable of delivering wavelengths of light for acne treatment and collagen building, yet suitably compact and safe.

### SUMMARY

Embodiments disclosed herein include devices and methods that can kill the bacteria that cause acne, as well as rebuild collagen to address dermatological issues of aging. Embodiments can contribute to skin brightening and tightening, reduction in size of skin pores, reduction of acne scarring, reduction of general scarring, reduction of blemishes and reduction of skin redness from irritation. Embodiments may also be useful for other photo-medicine applications.

Embodiments may include a single device for acne, a single device for anti-aging, a single device for other photo-medical use or a combination for acne and anti-aging and/or other photo-medical use. Devices can be indicated for use on face, back, arms, whole body, etc. Those skilled in the art will understand that devices can treat additional places and may be applicable to other ailments.

One embodiment can include an electrically powered device that exposes the skin surface to light emitted from light-emitting diode(s) contained within the device. In one embodiment, LEDs ranging from 350 nm to 500 nm may be used for anti-microbial treatments. LEDs of 600 nm to 1000 nm may be used for anti-inflammation and collagen growth. Multi-LED systems in various combinations and ratios may be used to address different skin conditions. The device can be stationary or can move. In one embodiment, the device is a handheld device that is moved long the surface of the skin to expose the skin to light.

In one embodiment, a photo-medicine device may include LEDs of different wavelengths. For example, some embodiments may have one or more LEDs of wavelengths below 500 nm and one or more LEDs of higher than 500 nm. In some embodiments, the photo-medicine device may include one or more 415 nm LED lights to match the absorption peak of acne vulgaris, and therefore kill the acne-causing bacteria. LEDs may also be provided which emit 660 nm light, which promotes collagen growth and therefore reduces inflammation of the infected area. Devices may contain LEDs emitting varied ratios of the aforementioned wavelengths or other wavelengths. For instance, one embodiment of the device may contain one (1) 415 nm LED to three (3) 660 nm LED, two (2) 415 nm LED to two (2) 660 nm LEDs or three (3) 415 nm LED to one (1) 660 nm LED. Another embodiment may be a system with all 415 nm LEDs. Yet another embodiment may be a system with all 660 nm LEDs. Other embodiments may also be possible.

According to example embodiments, devices, systems, and methods for photo-medicine are provided for. A photo-medicine device may include a housing having: a mounting member and an application member including an aperture. An LED array having at least one LED configured to emit light through the aperture at a first wavelength and at least one LED configured to emit light through the aperture at a second wavelength may be mounted to the mounting member. The LED array may be in thermal communication with the mounting member such that the housing functions as a heat sink for the LED array. In some embodiments, the first wavelength comprises approximately 415 nm and the second wavelength comprises approximately 660 nm. In some embodiments, the housing has a heat dissipation surface area of at least three square inches per LED watt.

A method for phototherapy in accordance with embodiments includes activating a photo-medicine device and determining if the photo-medicine device is positioned to begin therapy. If the photo-medicine device is positioned to begin therapy, light may be applied from an LED array at a predetermined intensity; a treatment timer may be activated; and a temperature of the photo-medicine device may be monitored. Application of light from the LED array may be ceased if the treatment timer runs out or the temperature of the photo-medicine device exceeds a predetermined threshold. In some embodiments, a housing of the photo-medicine device is configured to sink heat from the LED array and has a heat dissipation surface area of at least three square inches per LED watt. In some embodiments, a rest timer is provided which regulates an interval the LED array remains off after a treatment period has elapsed or expired.

In some embodiments, the LED array has at least one LED configured to emit light at a first wavelength and at least one LED configured to emit light at a second wavelength. In some embodiments, the first wavelength comprises approximately 415 nm and the second wavelength comprises approximately 660 nm.

In some embodiments, a system for phototherapy includes a photo-medicine device comprising an LED array having at least one LED configured to emit light at a first wavelength and at least one LED configured to emit light at a second wavelength; and a computing device communicatively coupled to the photo-medicine device, the computing device configured to transmit one or more activation codes to the photo-medicine device and receive treatment data from the photo-medicine device.

### BRIEF DESCRIPTION OF THE DRAWINGS

A more complete understanding of various embodiments of optical systems and devices and the advantages thereof may be acquired by referring to the following description, taken in conjunction with the accompanying drawings in which like reference numbers indicate like features and wherein:

FIGS. 1A-1C depict diagrammatic representations of an embodiment of a photo-medicine device;

FIG. 2 depicts a block diagram illustrating components of an embodiment of a photo-medicine device;

FIG. 3A depicts a diagrammatic representation of an example LED array for an embodiment of a photo-medicine device;

FIG. 3B depicts a diagrammatic representation of an example LED array positioned within an embodiment of a photo-medicine device;

FIG. 4 illustrates time vs. temperature rises for examples of embodiments of a photo-medicine device;

FIGS. 5A-5B depict a flowchart illustrating example operation of embodiments;

FIG. 6 depicts a diagram illustrating spectral distribution for an example embodiment of a photo-medicine device;

FIG. 7 is depicts a diagram illustrating an embodiment of a system including an example photo-medicine device; and

FIG. 8 depicts a flowchart illustrating example operation of an embodiment.

### DETAILED DESCRIPTION

The disclosure and various features and advantageous details thereof are explained more fully with reference to the exemplary, and therefore non-limiting, embodiments illustrated in the accompanying drawings and detailed in the following description. Descriptions of known starting materials and processes may be omitted so as not to unnecessarily obscure the disclosure in detail. It should be understood, however, that the detailed description and the specific examples, while indicating the preferred embodiments, are given by way of illustration only and not by way of limitation. Various substitutions, modifications, additions and/or rearrangements within the spirit and/or scope of the underlying inventive concept will become apparent to those skilled in the art from this disclosure.

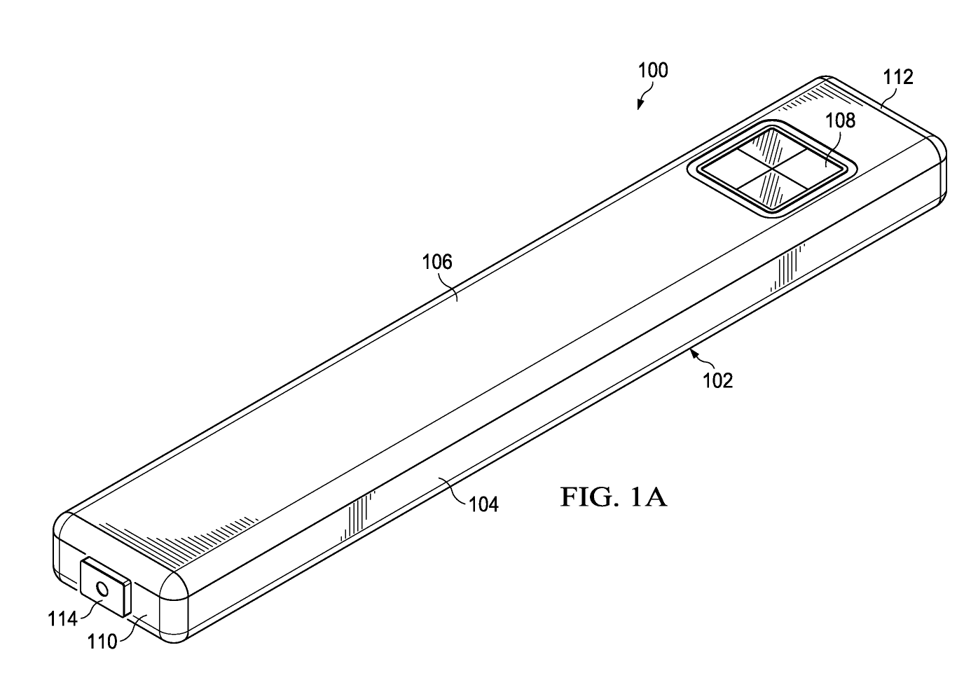

FIGS. 1A-1C illustrate an example of a photo-medicine device 100 in a perspective view, a front view, and a side view, respectively. In the example embodiment illustrated, the photo-medicine device 100 includes a housing 102 having a mounting member 104 and an application member 106. The application member 106 includes an aperture 108 through which light from an LED array may be emitted, as will be explained in greater detail below. In some embodiments, the application member 106 may be snap-fitted to the mounting member 104 to allow access to the interior of the device. In some embodiments, the photo-medicine device 100 may further include end plugs 110, 112. One of the end plugs 110 may include a receptacle for an electrical plug 114.

In some embodiments, the housing 102 may be formed of cast aluminum, extruded aluminum or other substance that provides suitable heat-sinking capabilities. The end plugs may be formed, e.g., of rubber or similar substance.

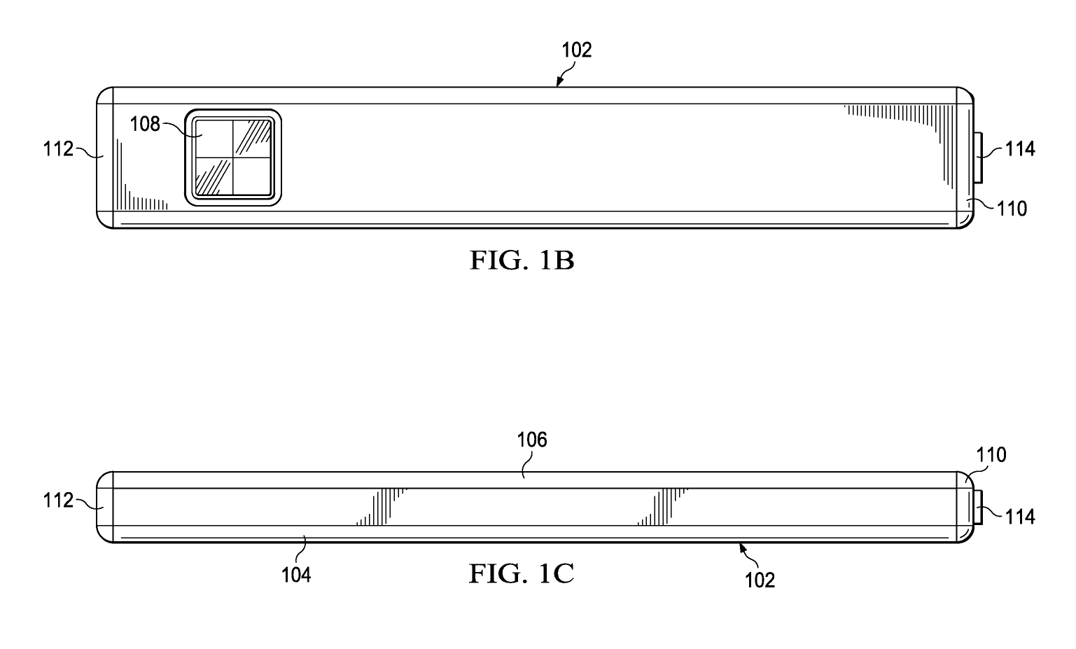

FIG. 2 is a block diagram of an example photo-medicine device 200. The photo-medicine device 200 may be an embodiment of the device shown in FIGS. 1A-1C. As shown, the photo-medicine device 200 includes a housing 202, an LED array 204, and an LED driver 206. As will be discussed in greater detail below, the LED array 204 may comprise an Aduro Surexi LED array, available from Illumitex, Inc. of Austin, Tex., U.S.A. Examples of systems and methods for making suitable LED arrays can be found in U.S. Pat. No. 7,772,604, issued on Aug. 10, 2010, entitled “SEPARATE OPTICAL DEVICE FOR DIRECTING LIGHT FROM AN LED” and U.S. Pat. No. 8,585,253, issued on Nov. 19, 2013, entitled “SYSTEM AND METHOD FOR COLOR MIXING LENS ARRAY,” both of which are incorporated by reference herein.

The photo-medicine device 200 may further include a controller 208, such as a microcontroller or microprocessor, and associated memory storing control instructions and/or data as will be explained in greater detail below. In general, the stored instructions can be executed to run various light recipes in therapy sessions to achieve desired fluence, application time, and/or spectral content. According to one embodiment, the recipes may be updated (e.g., by performing a firmware update through interaction with a computing device via various communications means such as Bluetooth, WiFi, infrared, radio frequency, etc.). Recipes may also be hard coded.

The photo-medicine device 200 may further include a user interface (UI) 210 and one or more sensors 212. The user interface 210 may include one or more manual or automatic control switches for turning the photo-medicine device on or off, dimming the LED array, and the like.

The user interface 210 may further include one or more control or status indicia, such as one or more LEDs or speakers to deliver alert sounds. Additionally, the user interface 210 may be capable of delivering one or more haptic indicia (i.e., vibrations) indicating device status. Finally, in some embodiments, the user interface may include a display or other indicator of one or more of power status, length of treatment time, overall usage time, battery charge level, and product life.

Sensors 212 may include, for example, capacitive sensors for detecting whether the photo-medicine device 200 is positioned close enough to the user's body to begin treatment (i.e., application of the LED light). Other sensors may include temperature sensors for monitoring the temperature of the device housing. In some embodiments, if the temperature exceeds a predetermined threshold, the device is turned off.

Photo-medicine device 200 may further include a timer (not shown) which is activated (e.g., by the controller 208) when the photo-medicine device 200 is activated or detected as having been moved into a treatment position. In some embodiments, when the timer reaches a predetermined count, the photo-medicine device 200 will become inactivated. In other embodiments, the timer may trigger an alert sound, vibration, or modulate the LED array 204 to provide a visual indicator.

Photo-medicine device 200 may further include a communication interface 214. The communication interface 214 may be one or more wired or wireless interfaces, such as USB, Bluetooth, WiFi, or infrared (IR) for communicating with other computing devices, such as laptop computers, personal computers, tablet computers, smartphones, and the like.

In some embodiments, the photo-medicine device 200 may transmit status indicators to the associated computing device. In some embodiments, such a computing device may transmit new LED recipes or instructions to the photo-medicine device 200.

In some embodiments, the photo-medicine device 200 may include a power supply 216. The power supply 216 may comprise rechargeable or nonrechargeable batteries and/or an AC power adapter.

In some embodiments, one or more arrays of LEDs that emit highly uniform blended light can be used for therapeutic purposes in the photo-medicine device 200. According to one embodiment, an LED array 204 may comprise an array of LEDs and an array of optical devices. An optical device can be configured to receive light from an LED and emit at least a majority (in some cases, at least 65%, at least 75%, at least 85%, at least 90%, at least 96%) of the light received from the LED in a desired half angle. In some cases, phosphor may be used. In some embodiments, the LED array can be an Aduro Surexi LED product by Illumitex, Inc. of Austin, Tex., with LEDs selected for emitting the desired wavelengths. For example, an Aduro Surexi LED (or other LED array) can be configured to emit light in a desired spectrum, as will be explained in greater detail below. The Aduro Surexi LED array can blend the varied wavelengths in a way that provides a relatively uniform treatment to the affected skin. The Aduro Surexi LED array also offers a powerful irradiance level that provides a relatively faster treatment protocol. It is noted that, while the photo-medicine device 200 of FIG. 2 includes a single array, devices may contain one, two, or more LED arrays to treat all or a portion of the body.

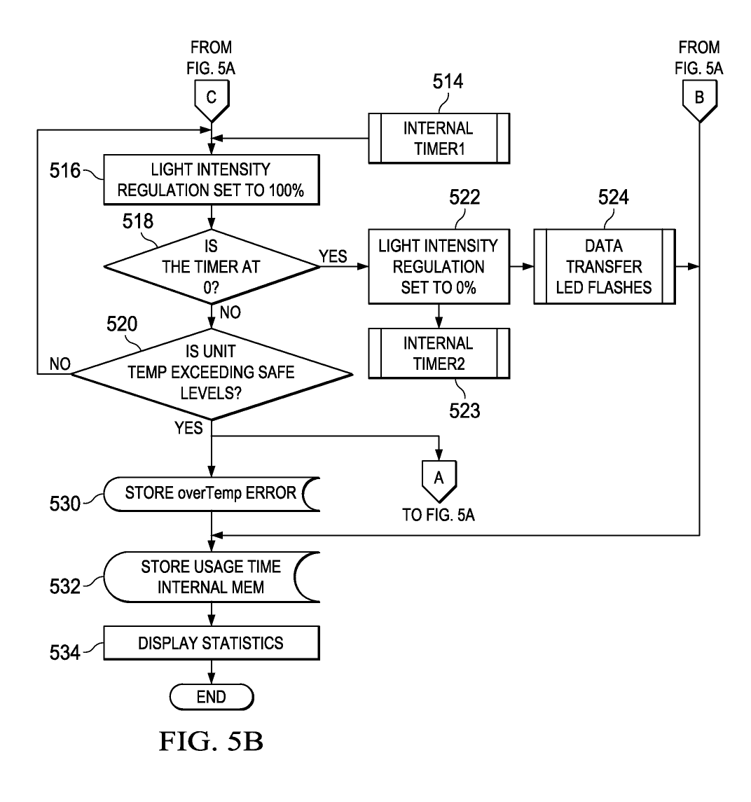

The LED source may be pulse width modulated or amplitude modulated to provide fluence levels down to 0 mW/cm² (fully dimmed) and up to 500 mW/cm². With an example spectrum as shown in FIG. 6 having peaks at approximately 415 nm and 660 nm, fluence levels of ˜400 mW/cm² may be achieved with the device proximate to the skin. The dosage levels may be as little as 1 J/cm² to 400 J/cm² for a 20 minute treatment. One embodiment uses fluence levels of 120 J/cm² for a five minute treatment. Those skilled in the art will understand that fluence level vs. time vs. spectral content can be optimized for a particular biological effect. In some embodiments, the array may include sixteen LEDs, including four (4) blue LEDs (˜450 nm) and twelve (12) red (˜660 nm) LEDs, although other ratios of red to blue are possible.

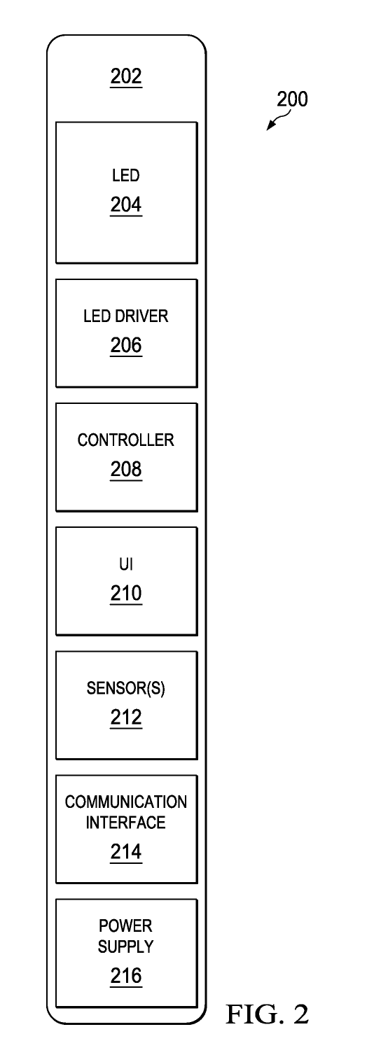

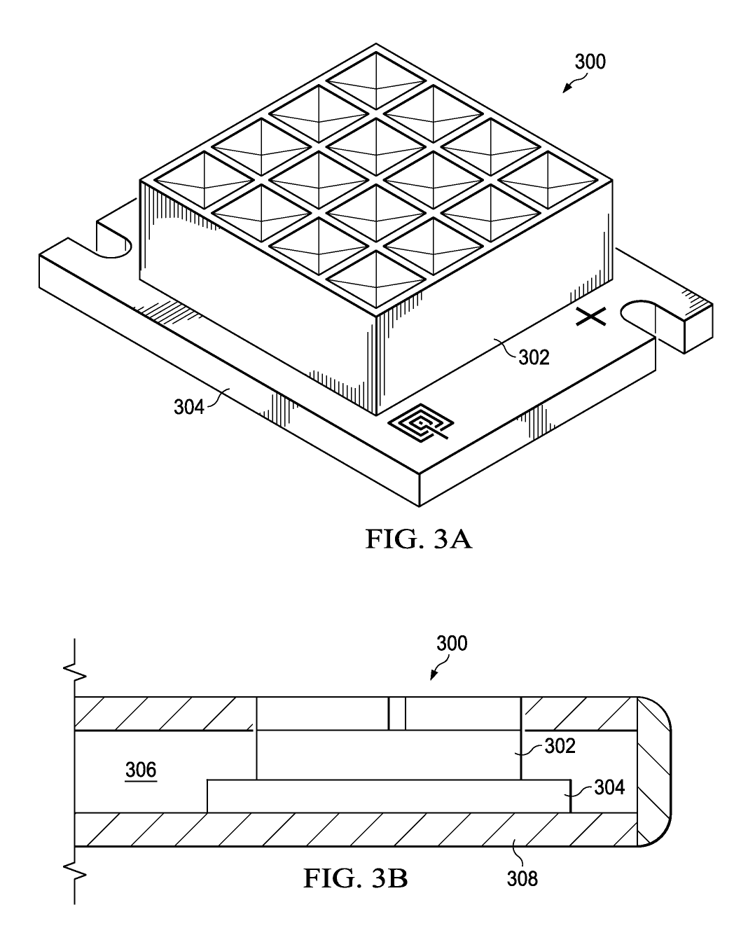

An example of a suitable LED array is shown in FIG. 3A. The array 300 includes

LEDs 302 and a mounting board 304 which functions as a heat sink. Advantageously, in one embodiment, the mounting board 304 is mounted to the mounting member 308 of the photo-medicine device 306. The mounting member then functions as a heat sink to transfer heat to the entirety of the device body, as shown in FIG. 3B.

As noted above, an important advantage of embodiments over prior photo-medicine devices is the relatively small, compact form factor. The minimum size and form factor of the device is constrained on the required heat dissipation of the LEDs and internal circuitry. Preferably, the minimum heat dissipation surface area is around 3 sq. inches per LED Watt. As noted above, heat from LEDs may be dissipated through the aluminum body and/or heat sink. In some embodiments the device may also incorporate an internal cooling fan. In other embodiments, a plastic housing may be employed, along with an internal heat capacitor (not shown).

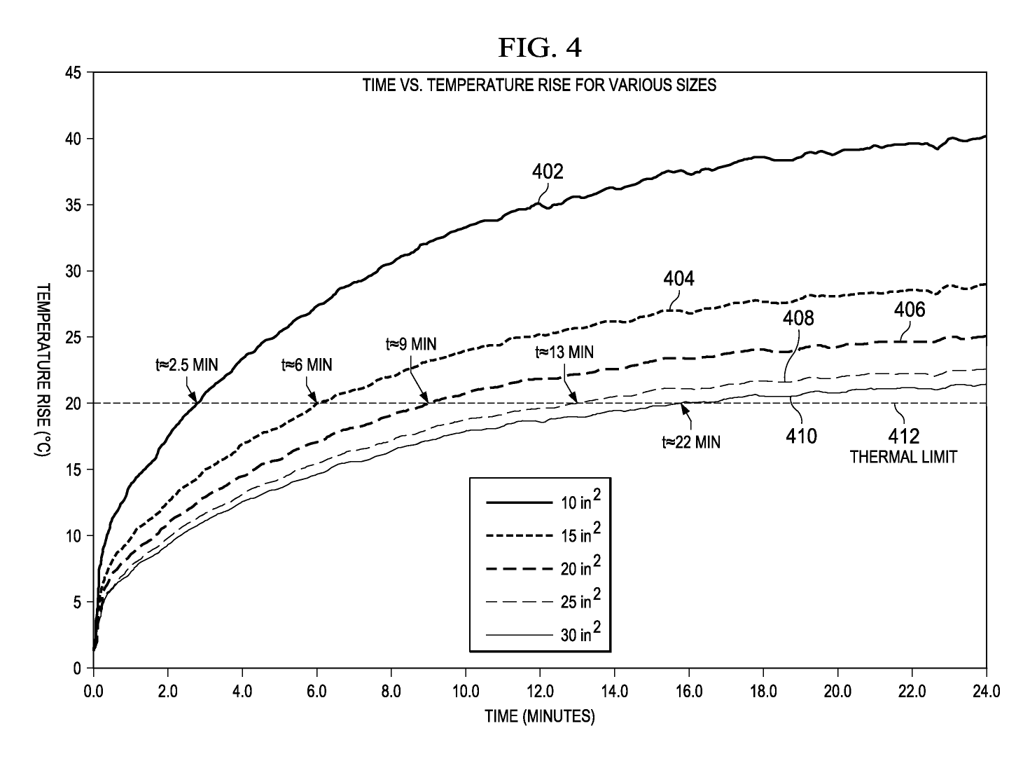

In embodiments in which a cast aluminum body is used to sink the heat, surface area of the body is an important parameter. For example, FIG. 4 shows time versus temperature rises for an extruded aluminum housing of varying sizes. Shown at 402 is a curve for a 10 square inch body; at 404 for a fifteen square inch body; and 406 for a 20 square inch body; at 408 for a 25 square inch body; and at 410 for a 30 square inch body.

Also shown in FIG. 4 is a thermal limit 412. In this example, the thermal limit 402 is arbitrarily set as a temperature change of 20 degrees Celsius, representing an amount most users would identify as getting “hot.”

As can be seen, the curve 402 crosses the thermal limit 412 at 2.5 minutes, the curve 404 crosses at 6 minutes; the curve 406 at 9 minutes; the curve 408 at 13 minutes; and the curve 410 at 22 minutes.

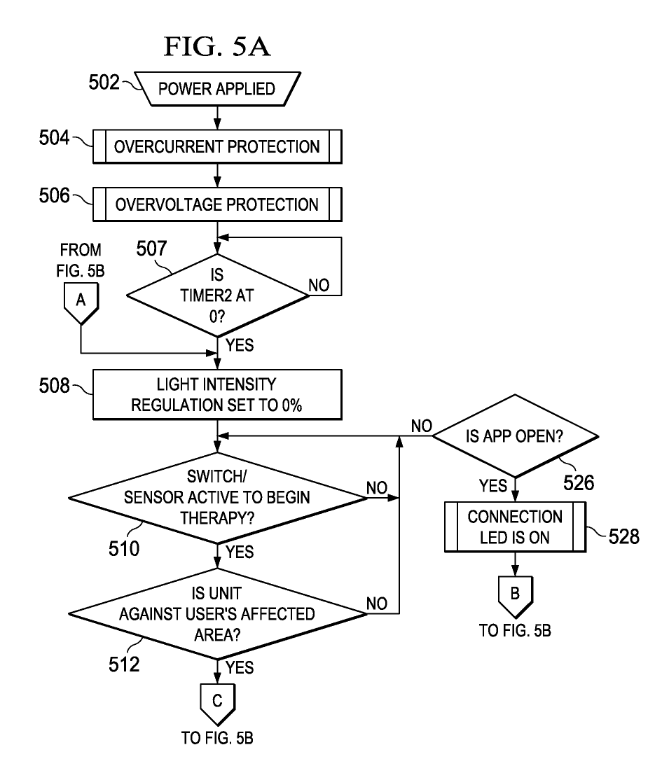

Turning now to FIGS. 5A and 5B, a flowchart illustrating operation of an embodiment is shown. At 502, power is applied to the photo-medicine device. As noted above, this may include activating a power switch to deliver battery or wall power to the device, or merely plugging the device into a wall outlet. In some embodiments, overcurrent protection 504 and overvoltage protection 506 may be provided. In some embodiments, a rest timer counts a predetermined time to keep the light off after the device times out or treatment otherwise ends; consequently, a check is made at 507 if the rest timer has expired.

Once power is applied, at step 508, the photo-medicine device controller functions to regulate light intensity, initially setting light intensity to 0%. Concurrently, the controller may monitor the communication interface to determine if an associated computing device is connected. For example, at step 526, the system may determine if a communication from a smartphone app has been received. If so, then in a step 528, a connection LED indicator may be activated.

At step 510, the controller determines if an appropriate interface member (e.g., a switch) or sensor (e.g., a capacitive proximity sensor and hence the photo-medicine device) has been activated or positioned (e.g., in proximity to a user's skin) to begin therapy. If not, the system cycles back to wait, as shown in FIG. 5A. If the controller determines (e.g., based on output from an interface member, a switch, or a sensor) that the photo-medicine device has been activated or positioned to begin therapy, in some embodiments, the controller may determine if the photo-medicine device is proximate to the affected area. Again, this determination may leverage output from a proximity sensor or other sensor. In embodiments in which this is determined, if the photo-medicine device is not against or proximate the user's affected area, then the system again cycles to wait, as shown in FIG. 5A.

If the photo-medicine device (also referred to herein as “unit”) is determined to be against or proximate the user's affected area, at 514, an internal treatment timer is started. As discussed above, such a treatment timer may be operable to run for a predetermined treatment time. In addition, at the same time, the light intensity is set by the controller to 100% at step 516. In some embodiments, the user interface or controls may include a dimmer wheel or other control for adjusting the 100% setting.

If the treatment timer has expired, as determined at step 518, then light intensity is set to 0 in step 522. In addition, in some embodiments, the rest timer may be activated to count a predetermined rest time, in a step 523. In some embodiments, at step 524, the LED array may flash to provide an indication of the termination of the treatment. Alternatively, aural or haptic indicia may be provided. In addition, in some embodiments, as will be explained in greater detail below, a data transfer may be made to a device such as a smartphone or personal computer.

If the treatment timer is still active, then in step 520, the system monitors the housing temperature of the unit. As discussed above, this may include the controller receiving a signal from a temperature sensor. If the temperature is not exceeding safe levels, then therapy is continued. If it is over safe levels, however, then light intensity is set back to 0%. In addition, an overtemperature error is stored at step 530, and a usage time is stored in a step 532. Finally, in embodiments in which a smartphone app is used, statistics may be transferred to the app for display at step 534.

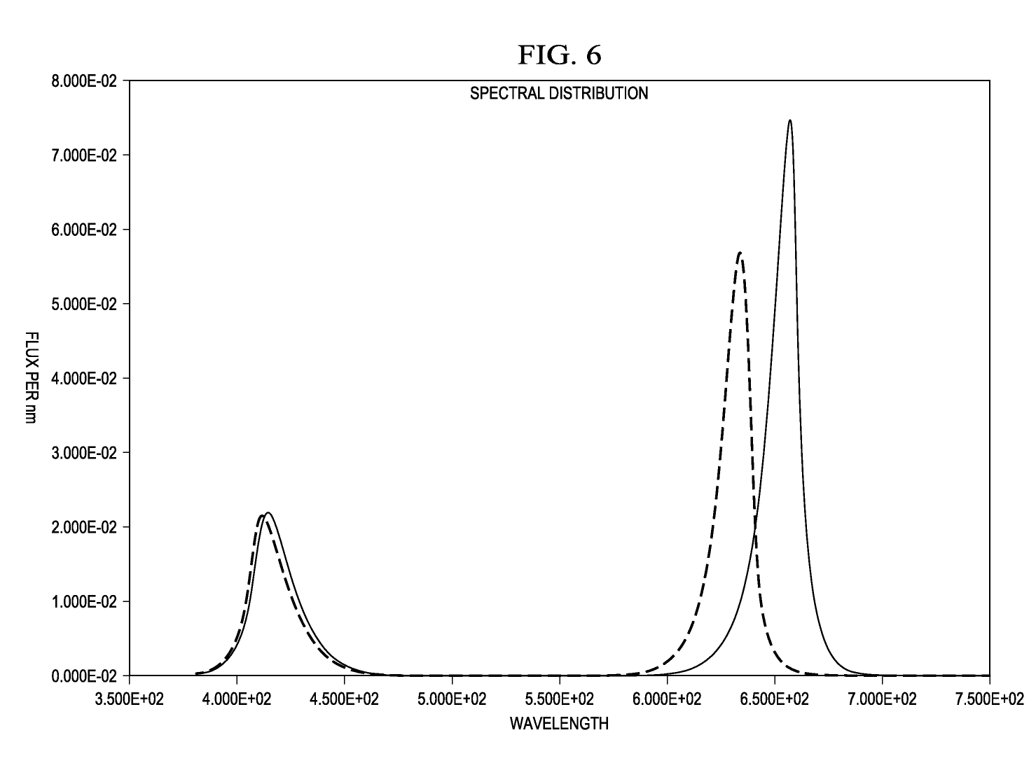

As noted above, in some embodiments, the photo-medicine device may be provided with a wireless communication interface for communicating with one or more computing devices over a network. For example, shown in FIG. 7 is a system 700 including a photo-medicine device 702, one or more networks 704, and computing devices 706a, 706b and 708. The networks 704 may be embodied as one or more WiFi, local area network (LAN), wide area network (WAN), the Internet, Bluetooth or other wireless network or networks.

The computing devices 706a, 706b may be embodied as personal or laptop computers, cellular telephones, table computers, and the like, typically owned by the user. In some embodiments, the computing devices 706a, 706b may send and receive commands and/or data to the photo-medicine device 702. The computing devices 706a, 706b may operate one or more applications or apps for interfacing with the photo-medicine device 702.

In some embodiments, the computing devices 706a, 706b may further be in communication with one or more servers 708. The one or more servers 708 may be in control of a provider of the photo-medicine device and may be used to send updates or activation codes to the photo-medicine device 702 via the network 704 and the computing devices 706a, 706b. In some embodiments, the photo-medicine device 70 may communicate directly with the server 708.

In some embodiments, the activation code may be valid for a predetermined period (e.g., one month) and may expire upon the end of that period. In this case, the user may be required to request a new authorization code via an app or web page maintained by the server 708. Such a request may include, for example, a payment of a subscription fee.

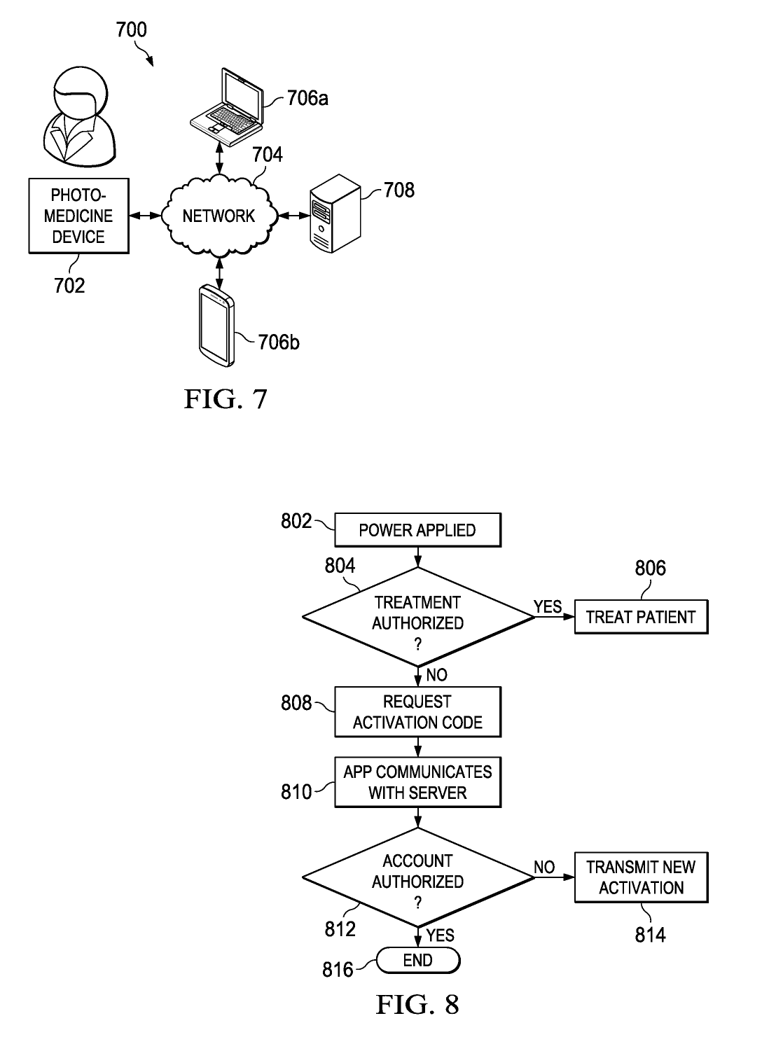

This process is shown with more particularity in FIG. 8. In a step 802, power is applied to the photo-medicine device 702. At step 804, the photo-medicine device controller may check if its activation for treatment is authorized. If so, then treatment may commence in a step 806 in the same manner or a similar manner as described above with reference to FIGS. 5A and 5B. If it is not authorized, however, then in a step 808, the photo-medicine device 702 may request authorization. For example, the photo-medicine device 702 may communicate with an app on a smartphone 706b via a WiFi or Bluetooth interface. At a step 810, the app or the photo-medicine device 702 may communicate with the server 708 to obtain the activation code. The server 708 may check a database or user profile to determine if the activation is authorized in a step 812. This may include, for example, receiving or checking if a payment has been received. If it has not, then in a step 816, the photo-medicine device 702 may remain inactive. Additionally, a non-activation message or payment reminder may be communicated (e.g., via the app) to the user. Otherwise, in a step 814, the new authorization code may be returned to the app and/or to the photo-medicine device itself.

Those skilled in the arts will appreciate after reading this disclosure that dimensions, materials, and other data provided herein are exemplary and that embodiments disclosed herein may be manufactured according to other dimensions, materials, or data without limiting the scope of the disclosure. Routines, methods, steps, operations or portions thereof described herein may be implemented through control logic, including computer executable instructions stored on a non-transitory computer-readable medium, hardware, firmware, or a combination thereof. The control logic can be adapted to direct a device to perform functions, steps, operations, methods, routines, operations or portions thereof described herein. Some embodiments may be implemented using software programming or code, application specific integrated circuits (ASICs), programmable logic devices, field programmable gate arrays (FPGAs), optical, chemical, biological, quantum or nanoengineered systems, components and mechanisms. Any suitable programming language may be used. Based on the disclosure and teachings provided herein, a person skilled in the art will appreciate other ways or methods to implement the invention.

A “computer-readable medium” may be any type of data storage medium that can store computer instructions, including, but not limited to read-only memory (ROM), random access memory (RAM), hard disks (HD), data cartridges, data backup magnetic tapes, floppy diskettes, flash memory, optical data storage, CD-ROMs, or the like. The computer-readable medium can be, by way of example, but not by limitation, an electronic, magnetic, optical, electromagnetic, infrared, or semiconductor system, apparatus, system, device, or computer memory. The computer-readable medium may include multiple computer-readable media storing computer executable instruction.

A “processor” includes any hardware system, hardware mechanism or hardware component that processes data, signals or other information. A processor can include a system with a central processing unit, multiple processing units, dedicated circuitry for achieving functionality, or other systems.

Embodiments of a photo-medicine device disclosed herein may be implemented to communicatively couple, via any appropriate electronic, optical, radio frequency signals, or other suitable methods and tools of communication in compliance with network or other communications protocols, to various computing devices and/or networks such as a personal computer, a database system, a smart phone, a network (for example, the Internet, an intranet, a local area network), etc. As is known to those skilled in the art, a computing device can include a central processing unit (“CPU”) or processor, memory (e.g., primary or secondary memory such as RAM, ROM, HD or other computer-readable medium for the persistent or temporary storage of instructions and data) and one or more input/output (“I/O”) device(s). The I/O devices can include a keyboard, monitor, printer, electronic pointing device (for example, mouse, trackball, stylus, etc.), touch screen, or the like.

As used herein, the terms “comprises,” “comprising,” “includes,” “including,” “has,” “having” or any contextual variant thereof, are intended to cover a non-exclusive inclusion. For example, a process, product, article, or apparatus that comprises a list of elements is not necessarily limited to only those elements but may include other elements not expressly listed or inherent to such process, product, article, or apparatus.

Further, unless expressly stated to the contrary, “or” refers to an inclusive or and not to an exclusive or. That is, the term “or” as used herein is generally intended to mean “and/or” unless otherwise indicated. For example, a condition A or B is satisfied by any one of the following: A is true (or present) and B is false (or not present), A is false (or not present) and B is true (or present), and both A and B are true (or present).

As used herein, a term preceded by “a” or “an” (and “the” when antecedent basis is “a” or “an”) includes both singular and plural of such term unless the context clearly dictates otherwise. Also, as used in the description herein, the meaning of “in” includes “in” and “on” unless the context clearly dictates otherwise.

Additionally, any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of ordinary skill in the art will appreciate that any term or terms with which these examples or illustrations are utilized will encompass other embodiments which may or may not be given therewith or elsewhere in the specification, and all such embodiments are intended to be included within the scope of that term or terms. Language designating such non-limiting examples and illustrations includes, but is not limited to: “for example,” “for instance,” “e.g.,” “in a representative embodiment,” “in one embodiment.”

Reference throughout this specification to “one embodiment,” “an embodiment,” “a representative embodiment,” or “a specific embodiment” or similar terminology means that a particular feature, structure, or characteristic described in connection with the embodiment is included in at least one embodiment and may not necessarily be present in all embodiments. Thus, respective appearances of the phrases “in one embodiment,” “in an embodiment,” or “in a specific embodiment” or similar terminology in various places throughout this specification are not necessarily referring to the same embodiment. Furthermore, the particular features, structures, or characteristics of any particular embodiment may be combined in any suitable manner with one or more other embodiments.

Although embodiments have been described in detail herein, it should be understood that the description is by way of example only and is not to be construed in a limiting sense. It is to be further understood, therefore, that numerous changes in the details of the embodiments and additional embodiments will be apparent, and may be made by, persons of ordinary skill in the art having reference to this description. The scope of the disclosure should be determined by the following claims and their legal equivalents.

What is claimed is:

1. A photo-medicine device, comprising:
a housing having a mounting member;
a controller comprising a processor, a non-transitory computer readable medium, and stored instructions translatable by the processor; and
a light-emitting diode (LED) array mounted to the mounting member, the LED array having:
at least one LED configured to emit light at a first wavelength; and
at least one LED configured to emit light at a second wavelength;
wherein the LED array is in thermal communication with the mounting member such that the housing functions as a heat sink for the LED array.

2. The photo-medicine device of claim 1, wherein the first wavelength comprises less than 500 nm and the second wavelength comprises greater than 500 nm.

3. The photo-medicine device of claim 1, wherein the first wavelength comprises approximately 415 nm and the second wavelength comprises approximately 660 nm.

4. The photo-medicine device of claim 1, wherein each of the first wavelength and the second wavelength comprises approximately 415 nm.

5. The photo-medicine device of claim 1, wherein each of the first wavelength and the second wavelength comprises approximately 660 nm.

6. The photo-medicine device of claim 1, wherein the housing has a heat dissipation surface area of at least three square inches per LED watt.

7. The photo-medicine device of claim 1, further comprising:
a treatment timer for regulating an application time of light emission from the LED array.

8. The photo-medicine device of claim 7, further comprising:
a rest timer for regulating an interval time that the LED array is off after the treatment timer has expired.

9. The photo-medicine device of claim 1, further comprising:
a temperature sensor for sensing a temperature of the housing.

10. The photo-medicine device of claim 1, further comprising:
a proximity sensor for sensing a closeness of the housing relative to a skin surface.

11. A method, comprising:
activating a photo-medicine device having a housing, a controller, a treatment timer, a rest timer, a temperature sensor, and a light-emitting diode (LED) array, the controller comprising a processor, a non-transitory computer readable medium, and stored instructions translatable by the processor, wherein the LED array is in thermal communication with the housing;
responsive to the activating, the controller determining whether the photo-medicine device is positioned to begin a therapy session on a skin surface;
when the photo-medicine device is positioned to begin the therapy session on the skin surface, the controller:
applying light from the LED array at a predetermined intensity;
activating the treatment timer for a predetermined count;
monitoring a temperature of the housing using the temperature sensor;
ceasing application of light from the LED array when the treatment timer runs out or when the temperature of the housing exceeds a predetermined threshold; and
activating the rest timer for regulating an interval time that the LED array is off for a predetermined period of time after the therapy session has ended.

12. The method according to claim 11, wherein the housing is configured to sink heat from the LED array and has a heat dissipation surface area of at least three square inches per LED watt.

13. The method according to claim 11, wherein the LED array has at least one LED configured to emit light at a first wavelength and at least one LED configured to emit light at a second wavelength.

14. The method according to claim 13, wherein the first wavelength comprises less than 500 nm and the second wavelength comprises greater than 500 nm.

15. The method according to claim 13, wherein the first wavelength comprises approximately 415 nm and the second wavelength comprises approximately 660 nm.

16. The method according to claim 13, wherein each of the first wavelength and the second wavelength comprises approximately 415 nm.

17. The method according to claim 13, wherein each of the first wavelength and the second wavelength comprises approximately 660 nm.

18. The method according to claim 11, further including transmitting treatment information from the photo-medicine device to a computing device.

19. The method according to claim 11, further including transmitting activation information from a computing device to the photo-medicine device.

20. A system for phototherapy, comprising:
a photo-medicine device including a light-emitting diode (LED) array having at least one LED configured to emit light at a first wavelength and at least one LED configured to emit light at a second wavelength; and
a computing device communicatively coupled to the photo-medicine device, the computing device configured to transmit one or more activation codes to the photo-medicine device and receive treatment data from the photo-medicine device.

21. The system of claim 20, wherein a housing of the photo-medicine device is configured to sink heat from the LED array and has a heat dissipation surface area of at least three square inches per LED watt.

22. The system of claim 20, wherein the first wavelength comprises less than 500 nm and the second wavelength comprises greater than 500 nm.

23. The system of claim 20, wherein the first wavelength comprises approximately 415 nm and the second wavelength comprises approximately 660 nm.

24. The system of claim 20, wherein each of the first wavelength and the second wavelength comprises approximately 415 nm.

25. The system of claim 20, wherein each of the first wavelength and the second wavelength comprises approximately 660 nm.

26. The system of claim 20, further comprising:
a treatment timer for regulating an application time of light emission from the LED array.

27. The system of claim 26, further comprising:
a rest timer for regulating an interval time that the LED array is off after the treatment timer has expired.

28. The system of claim 20, wherein the photo-medicine device further comprises a temperature sensor for sensing a temperature of the housing.

29. The system of claim 20, wherein the photo-medicine device further comprises a proximity sensor for sensing a closeness of the photo-medicine device relative to a skin surface.

30. The system of claim 29, wherein when the photo-medicine device is positioned to begin a therapy session on the skin surface, the photo-medicine device:
applying light from the LED array at a predetermined intensity;
activating a treatment timer for a predetermined count;
monitoring a temperature of the photo-medicine device;
ceasing application of light from the LED array when the treatment timer runs out or when the temperature exceeds a predetermined threshold; and
activating a rest timer for regulating an interval time that the LED array is off for a predetermined period of time after the therapy session has ended.
